(19) United States
(12) Patent Application Publication
(10) Pub. No.: US 2009/0148416 A1
(43) Pub. Date: Jun. 11, 2009

(54) ANGIOGENICALLY INDUCED TRANSPLANTS AND METHODS FOR THEIR USE AND MANUFACTURE

(76) Inventors: Ekaterina Vorotelyak, Moscow (RU); Andrey Vasiliev, Moscow (RU); Elina Chermnykh, Protvino (RU); Nikolai Tankovich, San Diego, CA (US)

Correspondence Address: STEMEDICA CELL TECHNOLOGIES, INC, 5375 MIRA SORRENTO PLACE, SUITE 100, SAN DIEGO, CA, 92121, US

(21) Appl. No.: 11/952,018

(22) Filed: Dec. 6, 2007

(51) Int. Cl. A61K 35/00; A61F 2/10

(52) U.S. Cl. 424/93.7; 623/15.12

(57) ABSTRACT

Angiogenically induced transplants and methods for their use and manufacture are disclosed. The angiogenic potential of the transplants is increased by contacting the transplants with donor mesenchymal cells such as hair follicle dermal papilla stem cells. Methods for treating disorders and diseases, such as disorders of the skin and angiopathies, are also disclosed.

### FIELD OF THE INVENTION

The present invention is in the field of tissue transplantation and the treatment of angiopathies. In particular, the invention relates to cell, tissue and organ transplants that have been angiogenically induced through the introduction of donor mesenchymal cells.

### BACKGROUND

Transplants offer promise in tissue healing and repair, and the replacement or treatment of diseased or dysfunctional tissues. A primary challenge in the transplantation of tissues is ensuring a sufficient blood supply to the transplant's constituent cells.

Ultimately, the success of a transplant depends on (a) a sufficient blood vessel density within the transplanted tissue or organ, (b) the organization of the vessels into a network comprised of low-resistance conduit vessels (arteries), (c) a functional microcirculation (arterioles and capillaries) for a proper blood-tissue exchange, and (d) functional drainage/compliance vessels (venules and veins). Transplants which do not undergo sufficient vascularization upon transplantation are subject to necrotization. Thus, in certain respects, the extent to which a transplant can become vascularized determines the limits of transplant success, as well as transplant size and architecture (see Oxford Textbook of Surgery, Morris and Malt, Eds., Oxford University Press (1994) and U.S. Pat. Nos. 5,432,053, 4,798,824, 4,879,283, 4,873,230, 5,405,742, 5,565,317, 5,370,989 and 5,552,267).

### SUMMARY OF THE INVENTION

The invention relates to angiogenically induced transplants and methods for their use in transplantation procedures and the treatment of angiopathies.

One objective of the invention is to provide an angiogenically induced transplant comprising a tissue explant suitable for treating a targeted disorder, and donor mesenchymal cells in contact with said tissue transplant.

A further objective of the invention is to provide an angiogenically induced transplant comprising a tissue explant suitable for treating a targeted disorder, and donor mesenchymal cells in contact with said tissue transplant.

A further objective of the invention is to provide an angiogenically induced transplant comprising a tissue explant suitable for treating a targeted disorder, and donor mesenchymal cells in contact with said tissue explant, wherein said explant is selected from the group consisting of a skin graft, a bone graft, a liver transplant, an eye graft, an eye transplant, a heart transplant, a blood vessel graft, a kidney transplant, a spleen transplant, a thymus transplant, a lung transplant, a digestive organ transplant, a tooth transplant, a nail transplant, a cartilage transplant, a hair transplant, and a reproductive organ transplant.

A further objective of the invention is to provide a method for treating a tissue disorder in a patient comprising administering to said patient an angiogenically induced transplant comprising a tissue explant and donor dermal papilla stem cells in contact with said tissue explant.

A further objective of the invention is to provide a method for treating an angiopathy in a tissue in a patient comprising contacting said tissue with an angiogenically induced transplant.

### BRIEF DESCRIPTION OF THE DRAWINGS

FIG. 1 is a drawing showing the structure of the hair follicle.

FIG. 2 is a micrograph depicting the in situ staining of hair follicle dermal papilla cells with toluidine blue.

FIG. 3 is a micrograph depicting the staining of cultured hair follicle dermal papilla cells with toluidine blue.

FIG. 4 is a micrograph depicting the in situ staining of hair follicle dermal papilla cells with aclian blue.

FIG. 5 is a micrograph depicting the staining of cultured hair follicle dermal papilla cells with aclian blue.

FIG. 6 is a micrograph depicting the staining of cultured hair follicle dermal papilla cells with alkaline phosphatase.

FIG. 7 is an ex vivo image of a non-angiogenically induced full thickness skin graft 3 weeks after transplantation into a nude mouse.

FIG. 8 is an image of a full thickness skin graft 3 weeks after transplantation into a nude mouse. The graft was injected with donor dermal papilla stem cells and donor keratinocytes.

FIG. 9 is an image of a full thickness skin graft 3 weeks after transplantation. The graft was injected with donor embryonic fibroblasts and donor keratinocytes.

### DEFINITIONS

As used herein, the phrase “stem cell” refers to an undifferentiated cell having the ability to both self-renew and differentiate to produce at least one functional, terminal cell type. One non-limiting example of a stem cell is a “precursor cell,” or “progenitor cell” which is a lineage-committed stem cell capable of dividing and differentiating to form a single, specific terminal cell type.

As used herein, the term “pluripotent,” or “pluripotency,” refers to the ability of a stem cell to form specialized cells belonging to any of the mesoderm, endoderm and ectoderm tissue lineages.

As used herein, the term “multipotent,” or “multipotency,” refers to the ability of a stem cell to form more than one cell type belonging to a single germ lineage (i.e. the endoderm, ectoderm or mesoderm). For example, a mesenchymal cell which has the ability to form chondrocytes, adipocytes and osteocytes is a multipotent mesenchymal cell.

As used herein, the phrase “mesenchymal cell” refers to cells belonging to (i.e. originating from) the mesodermal germ lineage. Mesenchymal cells may be multipotent stem cells, precursor cells, or fully differentiated terminal cells having a specific functional phenotype. Dermal papilla cells, dermal sheath cells, and fibroblasts are some non-limiting examples of mesenchymal cells.

As used herein, the term “donor” shall mean the source from which angiogenic mesenchymal cells and transplants (i.e. explants) are obtained. The donor and recipient of angiogenic mesenchymal cells and the transplants disclosed herein may be the same or different individuals. When used as an adjective (e.g. “donor mesenchymal cells,” “donor MSC”), the term “donor” refers to the source from which the mesenchymal cells are obtained. Cells derived from an in vitro culture of cells may be described as being obtained from the “donor” that provided the tissue from which the culture of cells was obtained.

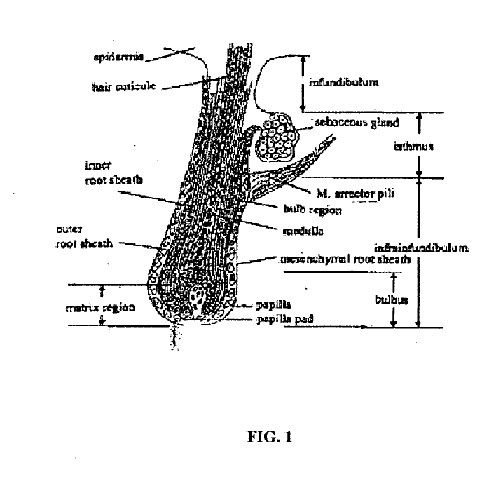

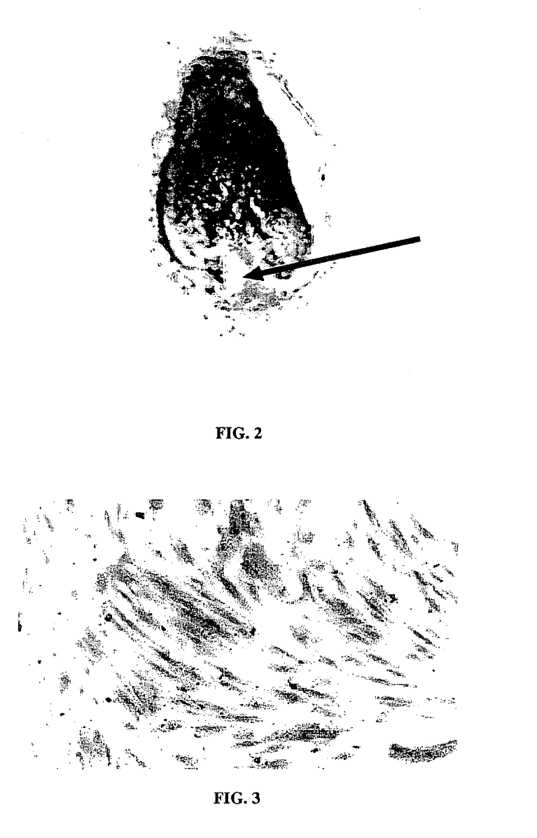

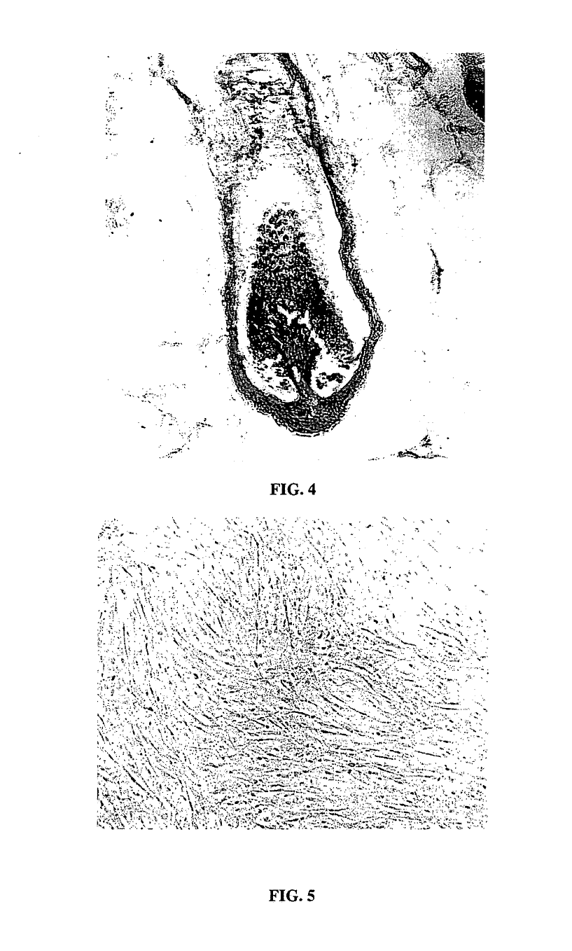

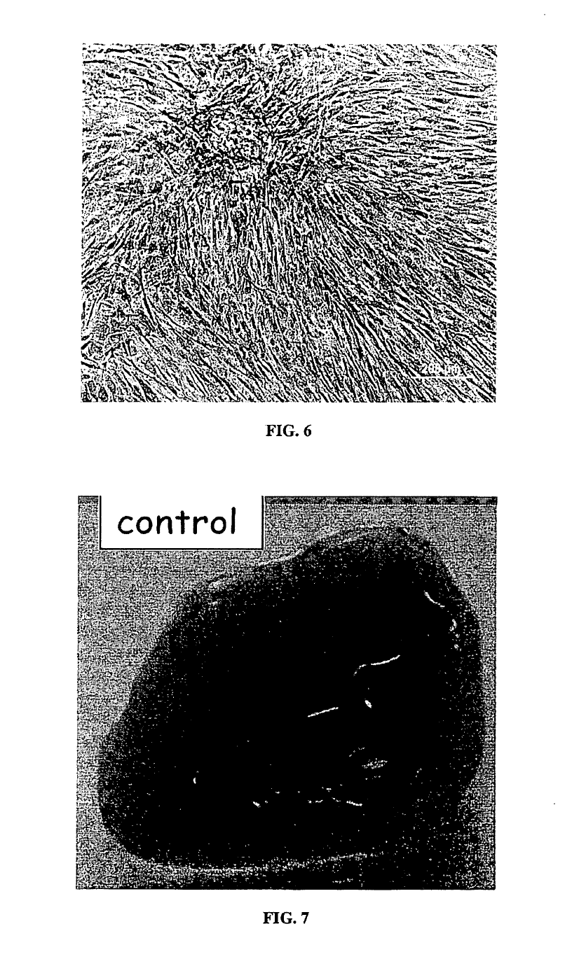

As used herein, the phrase “hair follicle cell” refers to any cell that originates from the structure of the hair follicle (see e.g. FIG. 1). Hair follicle cells may be hair follicle mesenchymal cells (HFMC) which include, but are not limited to, dermal papilla cells, dermal papilla stem cells, dermal sheath cells, and dermal sheath stem cells.

As used herein, the phrase “dermal papilla,” or “dermal papillae,” is used to refer to any of the superficial projections of the corium or dermis that interlock with recesses in the overlying epidermis that contain vascular loops and specialized nerve endings, and that are arranged in ridgelike lines. Dermal papilla cells may be found at the base of the hair follicle, and on the soles of the feet and palms of the hand.

As used herein, the phrase “dermal papilla cell” is used to refer to a cell that is derived from the dermal papilla. Dermal papilla cells include, but are not limited to, dermal papilla stem cells and the endothelial precursor cells which are associated with the endothelial tissue underlying the dermal papilla. A cell derived according to the method of Example 1 is one non-limiting example of a dermal papilla stem cell.

As used herein, the term “transplant,” or “explant,” refers to a tissue, organ, or collection of cells that has been separated from its natural environment. Transplants, include, but are not limited to, organs and tissues such as skin, kidney, heart, liver, lung, kidney, spleen, thymus, digestive organs, teeth, nails, hair, reproductive organs, as well as the functional parts of organs (e.g., segments of skin, sections of artery, transplantable lobes of a liver, kidney, lung, heart valves and the like).

Transplants further include populations of donor cells which have been separated from their natural environment, dissociated or otherwise processed, enriched or purified, and suspended, mixed or otherwise integrated in association with a pharmaceutically acceptable carrier. The transplants of the invention may be allogeneic, syngeneic or xenogeneic with respect to a transplant recipient.

As used herein the phrase “pharmaceutically acceptable carrier,” or “carrier,” means any of the well known liquid components useful for immunization such as, for example, culture media and phosphate-buffered saline. Some non-limiting examples of pharmaceutically acceptable carriers include, but are not limited to, those listed in Remington's Pharmaceutical Science (18.sup.th Ed., ed. Gennaro, Mack Publishing Co., Easton, Pa., 1990) and the Handbook of Pharmaceutical Excipients (4.sup.th ed., Ed. Rowe et al. Pharmaceutical Press, Washington, D.C.), each of which is incorporated by reference. “Pharmaceuticaly acceptable” means the carrier is non-toxic and does not cause an adverse reaction (e.g. an inflammatory or anergic reaction) when administered to a mammal.

As used herein, the term “matrix” refers to any non-cellular three dimensional material capable of supporting the growth of cells either in culture, or when the cells are transplanted in the body of a recipient. A matrix is considered to be a form of pharmaceutical carrier. Examples of suitable matrix materials include, but are not limited to sponges, gels, foams and combinations thereof. Matrices for use with the invention are disclosed in Jones et al. “A Guide to Biological Skin Substitutes” Brit J Plastic Surg (2002) 55, 185-193 which is incorporated herein by reference. Matrices may be made from a variety of materials including collagen, hyaluronic acid, chitin, silicone, polylactic acid, and combinations thereof.

As used herein, the phrase “suitable for treating a targeted disorder” refers to a substance (e.g. a transplant) that corrects, prevents, or reduces the pathological effect(s) of a disease, disorder, condition or injury, to a degree that is quantitatively or qualitatively measurable by one skilled in the art.

As used herein, the terms “treatment,” “treating,” “treat,” and the like, are used to generally mean obtaining a desired pharmacologic and/or physiologic effect against a targeted disease, disorder, condition or injury. The term “treat” may also refer to the angiogenic induction of a transplant (i.e. tissue explant) wherein angiogenic donor mesenchymal cells are contacted with the transplant.

As used herein the term, “isolated” refers to the separation of a substance (e.g. a cell) with a higher degree of purity than would be observed for the substance in its natural, unaltered state.

As used herein, the term “clone,” or “clonal cell,” refers to a single cell which is expanded to produce an isolated population of daughter cells (i.e. a “clonal cell population”).

As used herein, the phrase “cell line” refers to one or more generations of cells which are derived from a clonal cell.

As used herein, the phrase “derived from,” indicates that the cell came from a specific source such as, a tissue, a clonal cell line, a body fluid, body structure (e.g. hair follicle), or a primary cell culture.

As used herein, the term “angiogenesis” refers to a process of tissue vascularization that involves the development of new blood vessels. Angiogenesis occurs via one of three mechanisms: (1) “neovascularization,” where endothelial cells migrate out of pre-existing vessels to form new vessels; (2) “vasculogenesis,” where the vessels arise from precursor cells de novo; or (3) “vascular expansion,” where existing small vessels enlarge in diameter to form larger vessels (see e.g. Blood, C. H. and Zetter, B. R., 1990, Biochem. Biophys. Acta. 1032:89-118).

As used herein, the phrase “angiogenesis-dependent symptoms” refers to the symptoms of a disease or other pathological state that can be corrected, prevented or reduced by enhancing angiogenesis in a vascularly deficient tissue.

As used herein, the phrase “vascularly deficient tissue” refers to a tissue suffering from a pathology that adversely affects the development and/or maintenance of the vasculature of said tissue.

As used herein, the term “angiogenic” is used to describe a substance that has the ability to increase the rate (and/or overall amount of) angiogenesis in a material when the material is placed in the body of a recipient. The donor mesenchymal cells described herein are one example of an “angiogenic” substance in that they are capable of enhancing angiogenesis in an explant when the explant is (a) contacted with the donor mesenchymal cells, and (b) transplanted into the body of a recipient. Angiogenic substances include, but are not limited to angiogenic donor mesenchymal cells (e.g. donor dermal papilla stem cells),

The term “angiogenically induced” is used herein to describe a material has been contacted with an angiogenic substance. A transplant that has been contacted with donor angiogenic mesenchymal cells may be described as being “angiogenically induced.”

### DETAILED SPECIFICATION

The invention generally relates to transplants that are increased in their angiogenic potential through contact with angiogenic donor mesenchymal cells. Also disclosed and enabled are methods for using angiogenically induced transplants in transplant procedures and the treatment of angiopathies.

In some aspects of the invention, the angiogenically induced transplants comprise an explant (i.e. tissue, organ or collection of cells) that has been contacted with a preparation of angiogenic donor mesenchymal cells. Such angiogenically induced transplants undergo accelerated angiogenesis in the body of a patient leading to improvements in the transplant's establishment and success. The angiogenically induced transplants of the invention also find use in methods for treating angiopathies.

Angiogenic Donor Mesenchymal Cells

The transplants of the invention are angiogenically induced by contacting the transplants with angiogenic donor mesenchymal cells. Any preparation of donor mesenchymal cells can be used to prepare the transplants of the invention, provided that the introduction of the cells increases the angiogenic potential of the transplant upon implantation in the body of a transplant recipient.

Donor mesenchymal cells for use with the invention need not be derived from any particular tissue source provided that the mesenchymal cells are angiogenic. Thus, suitable donor mesenchymal cells may be syngeneic, allogeneic or xenogeneic with respect to the transplant with which they are contacted. It is also contemplated that the mesenchymal cells may be syngeneic, allogeneic or xenogeneic with respect to the patient that receives the transplant. Suitable donor mesenchymal cells may be derived from any animal, including, but not limited to, non-mammalian vertebrates (e.g., avian, reptile and amphibian) and mammals. In a preferred embodiment, the animal cells are mammalian cells and are derived from a mammal including, but not limited to, primates, mice, pigs, bovines, cats, goats, rabbits, rats, guinea pigs, hamsters, horses, sheep, or a combination thereof. In a more preferred embodiment, the cells are primate cells, for example, cells from humans, monkeys, orangutans, baboons, and combinations thereof. In a still more preferred embodiment, the donor mesenchymal cells are human cells.

Another aspect of the invention concerns the developmental stage of the donor source of the mesenchymal cells. The invention may be practiced with any age donor source provided that the donor mesenchymal cells obtained are capable of enhancing the angiogenic potential of a transplant. Suitable donor mesenchymal cells may therefore be derived from adult, fetal, embryonic or neonatal tissue sources. Mixed populations of angiogenic donor mesenchymal cells may also be derived from a combination of these sources.

Donor angiogenic mesenchymal cells for contacting with a transplant need not be derived from any particular tissue compartment. Accordingly, donor mesenchymal cells suitable for use with the invention may be derived from a variety of sources, including, but not limited to, hair follicle, bone marrow, adipose, peripheral blood, umbilical cord blood, umbilical cord, dermis, Wharton's jelly, periosteum, muscle tissue, uterine endometrium, amniotic fluid, tooth pulp, and combinations thereof.

Donor angiogenic mesenchymal cells may be derived from sources having varying degrees of purity. For example, donor mesenchymal cells may be purified from a tissue, derived from a primary culture of cells, derived from a clonal population of mesenchymal cells (i.e. a clonal cell line), or a combination thereof. The term “isolated,” or “purifed,” is used to describe a homogenous, or essentially homogenous, population of donor cells that has been separated from its natural environment, i.e the donor. A composition of cells is considered “purified,” or “substantially purified,” if it contains at least about 75%, at least about 80%, at least about 85%, at least about 90%, at least about 95% or at least about 100% of donor mesenchymal cells. A clonal population of donor mesenchymal cells is one non-limiting example of a purified cell preparation.

Donor angiogenic mesenchymal cell compositions may comprise purified or non-purified cell populations. Purified donor angiogenic mesenchymal cell compositions may be derived from clonal mesenchymal stem cells which have been expanded in culture. Alternatively, the purified cell composition may be derived from mesenchymal stem cells which have been isolated from a mixed population of cells, such as a fresh tissue preparation or a heterogeneous population of cells grown in culture. Methods for isolating donor mesenchymal cells from a mixed cell population are well known in the art and include, for example, FACS, cloning, density gradient centrifugation, magnetic sorting, affinity chromatography and serial passaging. Instructions for isolating mesenchymal stem cells suitable for practicing the invention are taught in the following references, the disclosures of which are incorporated by reference: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

Donor angiogenic mesenchymal cell compositions may also comprise a non-purified (i.e. mixed) population cells. Such a mixed cell composition may be obtained by, for example, combining two or more different purified mesenchymal cell types, or by culturing a mixed population of cells which has been expanded from a tissue sample (i.e. a primary cell culture). Suitable methods for preparing a primary culture of donor angiogenic mesenchymal cells are known in the art, and include, for example, those disclosed in Zhang et al; Cell Biol Int. Nov. 29, 2006, Sakaguchi et al. Arthritis Rheum. 2005 August;52(8):2521-9, Izadpana et al. J Cell Biochem. Dec. 1, 2006;99(5):1285-97, Mareschi et al. J Cell Biochem. Mar. 1, 2006;97(4):744-54, and Pozzi et al. Exp Hematol. 2006 July;34(7):934-42, each of which is incorporated by reference. It is also contemplated that the donor angiogenic mesenchymal cell compositions may be derived from an enriched population of mesenchymal cells obtained by the serial passage of a mixed population of cells such as a primary cell culture. Still further contemplated are mixed populations of cells obtained by combining an already mixed cell population with either one or more purified cell types, or a second mixed population of cells.

Purified donor angiogenic mesenchymal cells suitable for use with the invention are disclosed in the following publications, the disclosures of which are incorporated herein by reference: U.S. Pat. No. 5,654,186; U.S. Pat. No. 5,804,446; U.S. Pat. No. 5,980,887; U.S. Pat. No. 6,541,249; U.S. Pat. No. 6,676,937; U.S. Pat. No. 6,852,537; P.C.T. Pub. No. WO25112959; and U.S. Pat. Pub. No. 20060210544. As noted above, the purified cells referred to in these publications may be combined to form a non-purified composition of donor mesenchymal stem cells.

Depending on the size of a transplant, or the particular application to which the transplant is applied, it may be desirable to obtain a larger population of donor angiogenic mesenchymal cells. Accordingly, donor angiogenic mesenchymal cells may be expanded to provide a larger population of cells for treating large or multiple transplants. Donor angiogenic mesenchymal cells may be serially expanded according to the teachings of the following references, the disclosures of which are incorporated by reference: Wu et al., Arch Dermatol Res. 2005 August;297(2):60-7; Williams et al., Br J Dermatol. 1994 March; 130(3); Magerl et al. Exp Dermatol. 2002 August;11(4):381-5; Inamatsu et al., J Invest Dermatol. 1998 November 111(5):767-75; Warren et al., J Invest Dermatol. 1992 May;98(5):693-9.

In some aspects of the invention, donor angiogenic mesenchymal cells are derived from the hair follicle. Hair follicles suitable for use with the invention may be derived from any animal capable of providing hair follicle mesenchymal cells that increase the angiogenic potential of a transplant. In preferred embodiments, the hair follicles used are human. Such human hair follicles may be derived from any portion of the body, including, but not limited to the scalp and beard.

Donor hair follicle mesenchymal cells for increasing the angiogenic potential of transplants may be derived from one or more structures within the hair follicle, including a) the infundibulum: a section between hair follicle ostium and the intersection of the sebaceous gland into the hair canal; b) the isthmus: a section between the intersection of the sebaceous gland and the insertion of the M. arrector pili; c) the infrainfundibulum (suprabulbarportion): section between the insertion of the M. errector pili until the bulbus; and d) the hair bulbus (hair bulb) including the follicular dermal papilla.

In some aspects of the invention, hair follicle mesenchymal cells are derived from the hair bulb which is an onion-shaped structure located at the base of the growing hair follicle, and in the case of terminal hair, extends into the subcutaneous adipose tissue (see FIG. 1). Suitable angiogenic mesenchymal cells residing within the hair bulb include dermal papilla cells (Hardy (1992) Trends Genet. 8:55-61; Schmidt-Ullrich and Paus (2005) Bioessays 27:247-261). Dermal papilla cells for use with the invention may assume any level of plasticity so long as they are angiogenic. Thus, suitable angiogenic dermal papilla cells range from multipotent stem mesenchymal cells, down to fully differentiated terminal cells.

Angiogenic donor dermal papilla cells may be derived using any technique that yields cells capable of inducing angiogenesis in a transplant. In general terms, and without being limited to any particular theory, such hair follicle dermal papilla cells may be derived by taking haired skin and separating the epidermis and hair bulb by enzymatic digestion and mechanical separation using tweezers. The hair bulb is then further separated into dermal hair root sheath and hair dermal papilla. The hair dermal papilla is then subjected to a treatment with collagenase and trypsin to give single dermal papilla cells which are then labeled with a fluorescent dye (DiI) to give DiI-labeled hair DPC. The labeled dermal papilla cells may then be selected and grown in culture. Suitable methods for culturing dermal papilla cells for enhancing the angiogenic potential of transplants are readily available in the art as demonstrated by the following references, the disclosures of which are incorporated herein by reference: U.S. Pat. No. 5,851,831; U.S. Pat. No. 6,924,141; US 20070128172; US 20060088505; US 20040247573; Messenger et al. Brit. J. Derm. 110 (6), 685-689 (1984); WO/2006/124356; Randall et al. J. of Endocrinology, 133 (1) 141-147. Dermal papilla cell preparations for increasing the angiogenic potential of grafts may also be obtained commercially from sources such as PromoCell® (HFDPC-c, Cat. No. C-12071; HFDPC-p, C-12072).

In some aspects of the invention, angiogenic hair follicle dermal papilla cells are obtained from a fresh tissue preparation. Such embodiments may be practiced by purifying dermal papilla cells from a tissue preparation using markers such as, but not limited to, toluidine blue, alcian blue and alkaline phosphatase. It is also contemplated that hair follicle dermal papilla cells may be identified using biological markers including, but not limited to, smooth muscle alpha-actin, and prominin-1/CD133. Methods for purifying DPC through biological markers are taught in the following references, the disclosures of which are incorporated herein by reference: J Cell Sci. 1991 July;99 (Pt 3):627-36); and Ito et al., J Invest Dermatol. 2007 May; 127(5).

Dermal papilla cells for use with the invention may also be derived from the upper portion of the dermis (i.e. the papillary layer) which is characterized by tiny, fingerlike projections of tissue that indent into the epidermis above. Dermis particularly suited for providing such dermal papilla cells includes the thick skin on the palms and soles.

The invention contemplates priming the angiogenic mesenchymal cells of the invention prior to their being contacted with a transplant. One skilled in the art will appreciate the particular priming application that a selected transplantation procedure will require. Suitable methods for priming angiogenic mesenchymal cells disclosed herein include those taught in the following references, the disclosures of which are incorporated herein by reference: J Neuroimmunol. Nov. 1, 2000;111(1-2):177-85; Brain Res. Jul. 23, 2007;1159:67-76; J Neurochem. 2007 April;101(2):555-65. Primining of the angiogenic mesenchymal cells may take place by co-culturing the cells with astroglia or fibroblasts. It is also contemplated that the angiogenic mesenchymal cells may be primed by culturing the cells using fibroblast-conditioned or astroglial cell-conditioned medium. Angiogenic mesenchymal cells primed with astroglia cells or astroglia cell cultured medium may find use in treatment methods that address diabetic neuropathy.

Transplant Preparation

In some aspects of the invention, donor angiogenic mesenchymal cells (e.g. hair follicle dermal papilla stem cells) are used to increase the angiogenic potential of transplants. In such aspects a explant from a donor source is contacted with angiogenic mesenchymal cells under conditions which increases the angiogenic potential of the explant when it is transplanted in the body of a recipient, wherein the explant is an organ or tissue selected from, but not limited to, skin, bone, cartilage, liver, eye, endothelial tissue (e.g. veins capillaries or arteries), heart, kidney, spleen, thymus, lung, digestive organ, tooth, nail, hair and reproductive organs.

In some aspects of the invention, the angiogenically induced transplant is a donor or autologous skin graft. Such skin grafts may be split thickness or full thickness grafts, depending on the depth of the wound that the graft is being used to treat. Split skin grafts comprise the epidermis and a portion of the dermis and are obtained by shaving the surface of the skin with, for example, a dermatome. Patients suffering greater tissue loss may need a full-thickness skin graft, which includes the entire thickness of the skin. In this more complicated procedure, a flap of skin with its muscles and blood supply is transplanted to the area to be grafted. Common donor sites include skin and muscle flaps from the back or abdominal wall.

Transplants (i.e. explants) for use with the invention may be derived from a variety of donor sources and may be allogeneic (e.g. cadaver), syngeneic or xenogeneic with respect to the transplant recipient. A syngeneic transplant, for example, may be a skin graft wherein skin on a patient's body is relocated from one area to another.

In some aspects of the invention, the angiogenically induced transplant is a xenotransplant wherein the donor and recipient of the transplant are different species. Xenotransplants may be derived from any source that provides a transplant that is incorporated into, and accepted by, the body of the transplant recipient. Sources for xenotransplants for use with the invention include, but not limited to, non-mammalian vertebrates (e.g., avian, reptile and amphibian) and mammals. In a preferred embodiment, the transplant is derived from a mammal including, but not limited to, primates, mice, pigs, bovines, cats, goats, rabbits, rats, guinea pigs, hamsters, horses, sheep, and combinations thereof. Primate donor sources are the preferred donor source for many embodiments of the invention and include, for example, cells from humans, monkeys, orangutans, and baboons. In a preferred embodiment, the cells are human cells.

Transplants (i.e. organs, tissues, masses of cells, and/or isolated cells) for contacting with angiogenic mesenchymal cells can be harvested from a donor and transplanted by any method known to those of skill in the art (see Oxford Textbook of Surgery, Morris and Malt, Eds., Oxford University Press (1994)). The skilled practitioner will recognize that methods for harvesting and transplantation may vary depending upon many circumstances, such as the type of transplant and the condition to be treated in the recipient. In general terms, and without being limited to any particular theory, an organ is harvested under sterile conditions, cooled to about 4° C. and placed in a plastic bag submerged in a buffered salt solution containing nutrients. Methods for harvesting organ transplants, and methods for their short-term preservation, are taught in the following references, the disclosures of which are incorporated herein by reference: U.S. Pat. Nos. 5,432,053, 4,798,824, 4,879,283, 4,873,230, 5,405,742, 5,565,317, 5,370,989 and 5,552,267.

In some aspects of the invention, donor angiogenic mesenchymal cells are used to enhance the angiogenic potential of fresh transplants (e.g. fewer that 48 hours post-harvest). It is also contemplated that the transplants may be cryogenically preserved such as, for example, stored cadaver skin (see Atnip and Burke, 1983, Curr Prob. Surg. 20:623-83). Transplants may be contacted with donor angiogenic mesenchymal cells before, or after, cryopreservation. It is also contemplated that the invention may be practiced by contacting commercially available skin grafts with donor angiogenic mesenchymal cells. Suitable commercially available skin grafts for this purpose include, but are not limited to AlloDerm (LifeCell Corporation, Branchburg, N.J.) and Epicel® (Genzyme Corporation, Cambridge, Mass.).

One aspect of the invention concerns the manner in which the transplants are contacted with donor angiogenic mesenchymal cells. The transplant is contacted with donor mesenchymal cells in an amount sufficient to increase the angiogenic potential of the transplant. One skilled in the art will appreciate that this amount will vary with the type and size of the transplant, as well as the type of condition, disease or injury the transplant is being used to treat. The amount of donor mesenchymal cells necessary for enhancing angiogenesis will also vary according to the method in which the angiogenic mesenchymal cells are contacted with the transplant.

In one aspect of the invention, the angiogenic potential of a full-thickness skin graft is enhanced through the injection of an effective amount of donor dermal papilla stem cells. The cells are first prepared for injection by suspending a purified population of dermal papilla stem cells in a pharmaceutically acceptable carrier. Carriers suitable for this purpose include those taught in Remington's Pharmaceutical Science (18.sup.th Ed., ed. Gennaro, Mack Publishing Co., Easton, Pa., 1990) and the Handbook of Pharmaceutical Excipients (4.sup.th ed., Ed. Rowe et al. Pharmaceutical Press, Washington, D.C.), the disclosures of which are incorporated herein by reference. In some embodiments, the dermal papilla stem cells are suspended in phosphate buffered saline. In other embodiments, the dermal papilla stem cells are suspended in cell culture media. The preparation of donor dermal papilla stem cells may be injected at varying depths (e.g. intradermally), different volumes and different concentrations. In one aspect of the invention, the cells are injected between the dermis and epidermis in a sufficient volume to create a small reservoir of donor cells between, and in contact with one or both of the dermal and epidermal layers. Following the injection of the cells, the skin graft is ready for transplantation.

One aspect of the invention concerns the manner in which the angiogenically induced transplant is administered to the body of a patient. The particular procedure used will depend on the type of transplant that is being used and/or the type of angiopathy the transplant is intended to treat. Transplantation protocols known in the art are suitable and adaptable by one skilled in the art for use with the invention (see Oxford Textbook of Surgery, Morris and Malt, Eds., Oxford University Press (1994)).

Conditions Treated

The angiogenically induced transplants of the invention also find use in the treatment of a variety of circulatory disorders and diseases (i.e. angiopathies), including, but not limited to those relating to the skin, kidney, eye, endothelium (e.g. capillaries, arteries and veins), heart, liver, lung, spleen, thymus, digestive organs, teeth, nails, hair, reproductive organs. The invention also finds use in the treatment of the functional parts of organs (e.g., heart valves, segments of skin, sections of artery, transplantable lobes of a liver, kidney, lung, and the like).

In some aspects of the invention, angiogenically enhanced skin grafts are used to treat skin disorders and skin conditions including, but not limited to, wounds (e.g. burns), diabetic ulcers, infections causing skin loss, cosmetic replacement or reconstruction of skin, surgically removed gangrene, necrosis, lymphedema, skin grafts, wound healing and scleroderma.

The invention may also be used in the treatment of circulatory conditions that impair the vascularity of tissues and organs. As used herein, the term “vascularly impaired,” refers to a tissue or organ, that due to a disorder, disease, condition or injury, has decreased blood vessel function and/or blood vessel density in comparison to a healthy tissue or organ (i.e. a tissue or organ that is not affected by the disorder, disease, condition or injury).

One possible cause of a vascularly impaired tissue is chronic ischemic disease, such as after myocardial infarction or peripheral vascular disease. In such conditions, expansion of the vasculature adjacent to the affected tissue areas into the ischemic zones offers one mechanism by which these tissues can be recovered. Thus, implantation of an angiogenically induced transplant may act as a stimulus for revascularization of the affected areas. In this regard, the angiogenically induced transplant would act as a nucleus of vascular growth, rapidly establishing a new vascular network within the previously avascular or “hypovascular” zone. Implantation of angiogenically induced transplants not only provides for a rapid reperfusion of injured tissues, but also supports the restructuring and repair of those tissues.

Circulatory conditions and disorders which may be treated through the application of an angiogeneically induced transplant include, but are not limited to, the treatment of ischemic tissues and organs, tissues and organs having a zone or region that is avascular or hypovascular [e.g. chronic ischemic disease such as after myocardial infarction, peripheral vascular disease, or a cerbrovascular accident (stroke)], diabetic microangiopathy (e.g. diabetic retinopathy), revascularization of necrotic tissue, eye conditions (e.g., retinal neovascularization), and alopecia.

In practice, and in general terms, the transplants of the invention are used to treat a vascularly impaired tissue by first collecting a transplant, and preparing it by contacting with donor angiogenic mesenchymal cells (e.g. dermal papilla stem cells). The transplant is then combined, in the body of a patient, with a tissue or organ that is vascularly impaired and a revascularized tissue or organ is generated. As used herein, the term “combining” comprises contacting or implanting at least one angiogenically induced transplant on any surface, within, between the layers of, or adjacent to, a vascularly impaired tissue or organ. The angiogenically induced transplant can then encourage the development of a functional vascular bed with the surrounding functional vascular system leading to perfusion of the damaged tissue or organ.

The following examples illustrate the invention and are not to be understood as limiting the scope of the invention.

### EXAMPLE 1

### Isolation of Dermal Papilla Cells

Donor dermal papilla cells from hair follicle of human scalp skin were isolated by means of enzyme digestion. The scalp skin was normal skin obtained from a face-lift surgery. The skin was kept in Eagle's medium (containing antibiotics) at 4 degrees centigrade for not more than 48 hours. Skin specimens were first split at the dermis-subcutis interface by scalpel, then the dermis, containing hair follicles, was incubated in 0.5% dispase overnight. The dermal compartments of hair follicles were pulled out from cutaneous fat and the epithelium was teased out from the fibrous sheath with attached dermal papilla. Then the dermal sheaths were incubated in 0.1% collagenase in DMEM medium at 37 degrees centigrade for 6-8 hours until fibrous sheaths had been entirely digested, but just before the dermal papilla were digested. Dermal papilla were isolated completely out from the resuspension solution by repeated centrifugation.

The obtained cell culture was confirmed to be dermal papilla by testing with dermal papilla markers toluidine blue, alcian blue and alkaline phosphatase. Donor isolated cells were confirmed to have retained dermal papilla cell characteristics.

### EXAMPLE 2

### Preparation of Allogenic Grafts

a. Using Dermal Papilla Stem Cells Alone

Procedure

Human skin from face lift surgery was cut into 2 cm² pieces (i.e. grafts) and incubated in EDTA for 150 minutes. After incubation in EDTA, the skin grafts were ready for the injection of cells. All injections were made into a small pocket between the epidermis and dermis. The pocket was created by separating the dermis from the epidermis using a needle. 3 groups, consisting of 2 grafts each, were prepared as follows: Group 1 skin grafts formed the negative control group and these grafts received an injection of culture medium that was free of cells; Group 2 skin grafts formed the experimental group and these grafts received an injection of 5×10⁵ donor dermal papilla cells (from Example 1); and Group 3 skin grafts were used as a positive control and these grafts were injected with 5×10⁵ donor embryonic fibroblasts. Fibroblasts were used as a positive control due to their known angiogenic potential (see, e.g. J Dent Res. 2006 September;85(9):819-23). All cell injections, with the exception of the injection of embryonic fibroblasts, were conducted using cells which were derived from the same skin that was used to produce the skin grafts (i.e. using syngeneic cells). Injections were carried out using a volume of 50 microliters.

The skin grafts of Groups 1-3 were then transplanted on the backs of athymic mice. After 21 days, skin pieces were harvested and cryosected.

Results

Analysis of cyrosections revealed that 21 days after transplantation, Group 1 control grafts were crumpled and necrotized. In contrast, Group 2 grafts which were injected with donor dermal papilla cells were healthy, non-necrotized and permeated with blood vessels. Group 3 grafts were similarly healthy, non-necrotized and permeated with blood vessels. The fibroblasts of the Group 3 grafts were integrated among connective tissue fibers. Interestingly, both Group 2 and Group 3 grafts showed external hair fibers.

b. Using Dermal Papilla Stem Cells in Combination with Keratinocytes

Procedure

Human skin from face lift surgery was cut into 2 cm² pieces (i.e. grafts) and incubated in EDTA for 150 minutes. After incubation in EDTA, the skin grafts were ready for the injection of cells. All injections were made into a small pocket between the epidermis and dermis. The pocket was created by separating the dermis from the epidermis using a needle. 3 groups, consisting of 2 grafts each, were prepared as follows: Group 1 skin grafts formed the negative control group and these grafts received an injection of culture medium that was free of cells; Group 2 skin grafts formed the experimental group and these grafts received an injection of 2.5×10⁵ donor dermal papilla cells (from Example 1), and 2.5×10⁵ donor precursor keratinocytes; and Group 3 skin grafts were used as a positive control and these grafts were injected with 2.5×10⁵ donor embryonic fibroblasts and 2.5×10⁵ donor precursor keratinocytes. Fibroblasts were used as a positive control due to their known angiogenic potential (see e.g. J Dent Res. 2006 September;85(9):819-23). All cell injections, with the exception of the injection of embryonic fibroblasts, were conducted using cells which were derived from the same skin that was used to produce the skin grafts (i.e. using syngeneic cells). Injections were carried out using a volume of 50 microliters.

The skin grafts of Groups 1-3 were then transplanted on the backs of athymic mice. After 21 days, skin pieces were harvested and cryosected.

Results

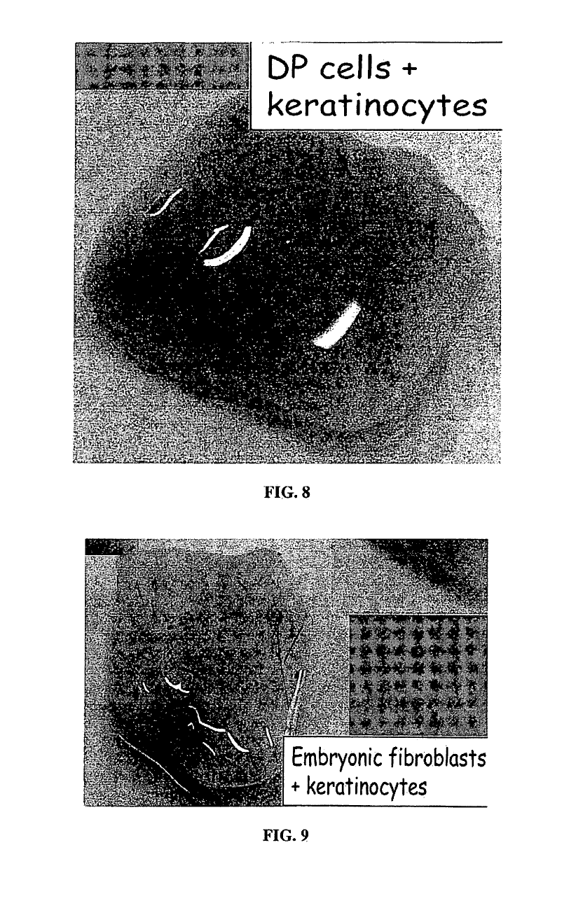

Analysis of cyrosections revealed that 21 days after transplantation, Group 1 control grafts were crumpled and necrotized (FIG. 7). In contrast, Group 2 grafts which were injected with donor dermal papilla cells were healthy, non-necrotized and permeated with blood vessels (FIG. 8). Group 2 grafts showed that the donor dermal papilla cells were well distributed among connective tissue fibers, while the donor keratinocytes localized in the basal membrane line (FIG. 9). Group 3 grafts were similarly healthy, non-necrotized and permeated with blood vessels (FIG. 9). Fibroblasts of Group 3 were integrated among connective tissue fibers. Interestingly, both Group 2 and Group 3 grafts showed external hair fibers, while the keratinocytes of the Group 2 grafts were found in the hair follicles.

### EXAMPLE 4

### Culture of Dermal Papilla Cells

Donor dermal papilla stem cells ordinarily undergo 3 culture passages before becoming terminally differentiated and losing their ability to divide. The inventor however has discovered a culture method and media formulation that provides for the extended culture of donor dermal papilla stem cells in an undifferentiated state. Using alkaline phosphatase as a marker for tissue precursor cells, the following experiment shows that the inventive culture method and media formulation increase the ability of donor dermal papilla stem cells to divide in an undifferentiated state.

Isolated donor dermal papilla cells were placed in plastic 6 well culture plates, averaging 7-10 dermal papilla cells per well. The medium was non-knockout DMEM medium (Sigma), supplemented with 10% fetal bovine serum (Biolot, St.-Petersburg). The cultures were kept untouched for five to six days. After this, the medium was refreshed every third day. Group 2 cells were used as the experimental group and after attachment of dermal papilla, the media was replaced with the inventive medium [i.e. ES-medium consisting of Knockout DMEM (Gibco) with addition of 6.5% Knockout serum replacement (Gibco), 6.5% ES qualified FBS (Gibco), non-essential amino acids (Gibco), 0.1 mM beta-mercaptoethanol, 20 ng/ml leukemia inhibitory factor (LIF) and 25 ng/ml basic fibroblast growth factor (bFGF)]. Group 1 cells were maintained as a control arid kept in the non-knockout DMEM medium.

In general, most of the donor dermal papilla cells attached to the plastic after 2-5 days of cultivation and achieved a cell monolayer after 2 weeks. After this, cells were passaged. Donor dermal papilla cells incubated in DMEM medium had a flattened morphology. Group 1 cells were negative for alkaline phosphatase after 3 passages in contrast to the dermal papilla cells cultivated in knockout DMEM medium. Group 2 cells (dermal papilla cells in knockout DMEM medium) showed positive alkaline phosphatase results for up to 7 passages. Dermal papilla cells cultivated in knockout DMEM medium also had different morphology being more stretched and narrow. Results of this experiment are summarized in the following table.

| Group | Medium | Results |
|---|---|---|
| 1 | DMEM medium (Sigma), | Cells had flattened |
| | supplemented with 10% | morphology and were |
| | fetal bovine serum (Biolot, | negative for alkaline |
| | St.-Petersburg) | phosphatase after 3 |
| | | passages. |
| 2 | Knockout DMEM (Gibco) | Cells had stretched and |
| | with addition of 6.5% | narrow morphology and |
| | Knockout serum | were positive for alkaline |
| | replacement (Gibco), 6.5% | phosphatase for up to 7 |
| | ES qualified FBS (Gibco), | passages. |
| | non-essential amino acids | |
| | (Gibco), 0.1 mM beta- | |
| | mercaptoethanol, 20 ng/ml | |
| | leukemia inhibitory factor | |
| | (LIF) and 25 ng/ml basic | |
| | fibroblast growth factor | |
| | (bFGF). | |

The Group 2 cells grown in the inventive media expressed alkaline phosphatase for up to 7 passages which was 4 more passages than that observed for the control Group 1 cells. Alkaline phosphatase expression serves as a marker for the ability of donor stem cells to divide and differentiate (Pera et al. J Cell Sci (2000) 113, 5-10). Thus, the inventive media and its method of use provides a means for the extended culture of donor dermal papilla stem cells in an undifferentiated state.

### EXAMPLE 5

### Transplantation of Skin Graft

Full thickness skin grafts obtained from face lift surgery are prepared for injection with the donor dermal papilla stem cells of Example 1 by incubating the graft in EDTA for 150 minutes. The donor dermal papilla stem cells are allogeneic with respect to both the skin graft, and allogeneic to the patient that receives the skin graft. Dermal papilla stem cells are prepared for injection into the experimental grafts by suspending the cells in DMEM medium with glutamin. The cell suspension is brought up to volume providing a cell density of 10⁷ cells/ml. The dermal papilla stem cells are then injected in between the epidermis and dermis at multiple spots on the experimental grafts allowing the dermal papilla stem cell suspension to form small pockets under the epidermis. The cell preparations are injected at a rate of 5×10⁵ donor dermal papilla cells/100 mm² of skin graft with an approximate volume of 50 microliters cell suspension being injected at each injection site.

Following the injection of the donor dermal papilla stem cells, the skin grafts are transplanted to human patients by suitable procedures as taught in Oxford Textbook of Surgery, Morris and Malt, Eds., Oxford University Press (1994). Control skin grafts are prepared and transplanted to patients in the same manner as the experimental grafts, except that the control grafts are not injected with dermal papilla stem cells.

The experimental grafts will show increased graft acceptance by the patient, and an increased blood vessel development, when compared to the control grafts which lack donor dermal papilla stem cells.

I claim:

1. An angiogenically induced transplant comprising:
a. a tissue explant; and
b. donor mesenchymal cells in contact with said tissue explant.

2. The angiogenically induced transplant of claim 1, wherein said mesenchymal cells are hair follicle mesenchymal cells (HFMC).

3. The angiogenically induced transplant of claim 2, wherein said HFMC are dermal papilla stem cells.

4. The angiogenically induced transplant of claim 1, wherein at least a portion of said mesenchymal cells are allogeneic with respect to said tissue explant.

5. The angiogenically induced transplant of claim 1, wherein at least a portion of said mesenchymal cells are autologous with respect to said tissue explant.

6. The angiogenically induced transplant of claim 1, wherein said tissue explant is derived from a human source.

7. The angiogenically induced transplant of claim 1, wherein said tissue explant is selected from the group consisting of a skin graft, a bone graft, a liver transplant, a heart transplant, a kidney transplant, a spleen transplant, eye graft, eye transplant, endothelial tissue transplant, a thymus transplant, a lung transplant, a digestive organ transplant, a tooth transplant, a nail transplant, a cartilage transplant, a hair transplant, and a reproductive organ transplant.

8. The angiogenically induced transplant of claim 4, wherein said mesenchymal cells are injected into said tissue explant.

9. The angiogenically induced transplant of claim 6, wherein said tissue explant is a skin graft.

10. The angiogenically induced transplant of claim 1, wherein said tissue explant is capable of promoting angiogenesis in a site in the body of a patient.

11. A method for preparing a tissue for administration to a subject comprising:
providing said tissue from a donor source; and
contacting said tissue with angiogenic mesenchymal cells;
wherein said mesenchymal cells promote angiogenesis in said tissue upon transplantation to the body of said subject.

12. The method of claim 10, wherein said mesenchymal cells are hair follicle mesenchymal cells (HFMC).

13. The method of claim 11, wherein said HFMC are dermal papilla stem cells.

14. The method of claim 10, wherein said mesenchymal cells are allogeneic with respect to said tissue.

15. The method of claim 10, wherein said mesenchymal cells are autologous with respect to said tissue.

16. The method of claim 10, wherein said tissue is selected from the group consisting of a skin graft, a bone graft, a liver transplant, a heart transplant, a kidney transplant, a spleen transplant, eye graft, eye transplant, endothelial tissue transplant, a thymus transplant, a lung transplant, a digestive organ transplant, a tooth transplant, a nail transplant, a cartilage transplant, a hair transplant, and a reproductive organ transplant.

17. The method of claim 13, wherein said mesenchymal cells are contacted with said tissue by injection.

18. The method of claim 14, wherein said tissue comprises a skin graft.

19. The method of claim 10, wherein said mesenchymal cells are contacted with to said tissue before said tissue is collected from said donor source.

20. The method of claim 10, wherein said mesenchymal cells comprise a dermal papillae stem cell extract.

21. The method of claim 10, wherein said mesenchymal cells promote angiogenesis at a site in the body of said subject.

22. The method of claim 18, wherein said site comprises a devascularized tissue.

23. The method of claim 18, wherein said site comprises a vascularly impaired tissue.

24. A method for treating a skin condition in a site on the skin of a patient comprising:
a. providing a skin graft from a donor source;
b. contacting said skin graft with angiogenic mesenchymal cells to produce an angiogenically induced skin graft;
c. applying said angiogenically induced skin graft to said site on said patient.

25. The method of claim. 24, wherein said angiogenic mesenchymal cells comprise dermal papilla stem cells.
